(19) United States
(12) Patent Application Publication
(10) Pub. No.: US 2026/0020987 A1
(43) Pub. Date: Jan. 22, 2026

(54) SWITCHABLE DRESSING FOR NEGATIVE-PRESSURE THERAPY

(71) Applicant: SOLVENTUM INTELLECTUAL PROPERTIES COMPANY, Maplewood, MN (US)

(72) Inventors: Andrew J. MCGREGOR, San Antonio, TX (US); Shannon C. INGRAM, San Antonio, TX (US); Benjamin A. PRATT, Poole (GB)

(21) Appl. No.: 18/993,305

(22) PCT Filed: Jul. 12, 2023
(86) PCT No.: PCT/IB2023/057170

§ 371 Date: Jan. 10, 2025

Related U.S. Application Data
(60) Provisional application No. 63/389,201, filed on Jul. 14, 2022.

(51) Int. Cl. A61F 13/05; A61F 13/0246; A61M 1/00; A61N 5/06

(52) U.S. Cl. CPC A61F 13/05; A61F 13/0246; A61M 1/91; A61N 5/0616; A61M 2205/052; A61M 2205/053; A61M 2210/04; A61N 2005/063; A61N 2005/0645; A61N 2005/0659; A61N 2005/0661

(57) ABSTRACT

Apparatuses, systems, and methods for treating a tissue site. A system for treating a tissue site with negative pressure includes a dressing and a dressing interface. The dressing includes a cover configured to be disposed over the tissue site, a tissue interface configured to be positioned adjacent to the cover, and a wound contact layer configured to be positioned adjacent to the tissue interface. The dressing interface is configured to be coupled to the cover to fluidly couple the tissue interface and the wound contact layer to a therapy unit. The dressing interface is also configured to transmit electromagnetic radiation through the cover.

### CROSS-REFERENCE TO RELATED APPLICATIONS

This application is a U.S. National Stage Entry of PCT International Application No. PCT/IB2023/057170, filed on Jul. 12, 2023, which claims the benefit of priority to U.S. Provisional Application No. 63/389,201, filed on Jul. 14, 2022, which is incorporated herein by reference in its entirety.

### TECHNICAL FIELD

The invention set forth in the appended claims relates generally to tissue treatment systems and more particularly, but without limitation, to systems, apparatuses, and methods for a light-switchable dressing for use with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for treating a tissue site in a negative-pressure therapy environment are set forth in the appended claims. Illustrative embodiments are also provided to enable a person skilled in the art to make and use the claimed subject matter.

For example, in some embodiments, a system for treating a tissue site with negative pressure is described. The system can include a dressing and a dressing interface. The dressing can include a cover configured to be disposed over the tissue site, a tissue interface configured to be positioned adjacent to the cover, and a wound contact layer configured to be positioned adjacent to the tissue interface. The dressing interface can be configured to be coupled to the cover to fluidly couple the tissue interface and the wound contact layer to a therapy unit. The dressing interface can further be configured to transmit electromagnetic radiation through the cover.

In some example embodiments, the cover can be configured to block ambient electromagnetic radiation.

In some example embodiments, the wound contact layer can include a lightguide and a switchable adhesive. The lightguide can be configured to receive and distribute the electromagnetic radiation from the dressing interface. The lightguide can include a first surface of the wound contact layer. The switchable adhesive can be configured to transition between a first tack and a second tack in response to exposure to the electromagnetic radiation from the dressing interface. The switchable adhesive can include a second surface of the wound contact layer. The second surface of the wound contact layer can be opposite the first surface of the wound contact layer. In some example embodiments, the lightguide can be configured to distribute the electromagnetic radiation from the dressing interface to the switchable adhesive. In some example embodiments, the electromagnetic radiation can include a first electromagnetic radiation. The switchable adhesive can be further configured to transition from the second tack to a third tack in response to exposure to a second electromagnetic radiation from the dressing interface. In some example embodiments, the lightguide can be further configured to distribute a therapeutic electromagnetic radiation to the tissue site

In some example embodiments, the wound contact layer further can further include a central portion and a periphery. The tissue interface can be configured to be positioned adjacent to the central portion of the wound contact layer. In some example embodiments, the dressing interface can include a first opening and a second opening. The first opening can be configured to be fluidly coupled to the tissue interface and the second opening can be configured to couple to the periphery of the wound contact layer.

In some example embodiments, the system can further include a fiber-optic cable configured to couple the dressing interface to the therapy unit.

In some example embodiments, the cover can be configured to be removable from the dressing.

Also described herein is a dressing for treating a tissue site. The dressing can include a sealing member, a manifold, and a distribution layer. The sealing member can be configured to be disposed over the tissue site. The manifold can be configured to be positioned adjacent to the sealing member. The distribution layer can include an electromagnetic transmission layer and an adhesive layer. The electromagnetic transmission layer can be configured to receive and distribute electromagnetic radiation. The adhesive layer can be configured to transition between a first tack and a second tack in response to exposure to electromagnetic radiation distributed by the electromagnetic transmission layer.

Also described herein is a method of treating a tissue site, the method can include placing a dressing adjacent to the tissue site. The dressing can include a cover configured to be disposed over the tissue site, a tissue interface configured to be positioned adjacent to the cover, and a wound contact layer configured to be positioned adjacent to the tissue interface. The wound contact layer can include a lightguide configured to receive and distribute electromagnetic radiation and a switchable adhesive configured to transition between a first tack and a second tack in response to exposure to the electromagnetic radiation. In some example embodiments, the method can further include coupling a dressing interface to the dressing. The dressing interface can be configured to deliver negative pressure and electromagnetic radiation to the dressing. The method can further include delivering a first electromagnetic radiation to the wound contact layer through the dressing interface and operating an electromagnetic radiation source to deliver a second electromagnetic radiation to the wound contact layer through the dressing interface.

In some example embodiments, delivering the first electromagnetic radiation to the wound contact layer can cure the switchable adhesive from the first tack to the second tack.

In some example embodiments, the second electromagnetic radiation can be a therapeutic electromagnetic radiation configured to treat the tissue site.

In some example embodiments, the method can further include operating the electromagnetic radiation source to deliver a third electromagnetic radiation to the wound contact layer. The third electromagnetic radiation can be configured to switch the switchable adhesive from the second tack to a third tack. In some example embodiments, the method can further include removing the dressing from the tissue site when the switchable adhesive has the third tack. In some example embodiments, the wound contact layer can further include a central portion and a periphery. The tissue interface can be configured to be positioned adjacent to the central portion of the wound contact layer. In some example embodiments, coupling the dressing interface to the dressing can include disposing a first opening of the dressing interface adjacent to the tissue interface and disposing a second opening of the dressing interface adjacent to the periphery of the wound contact layer.

In some example embodiments, the method can further include removing the cover from the dressing to expose the dressing to ambient electromagnetic radiation. The switchable adhesive can be configured to switch from the second tack to a third tack when exposed to the ambient electromagnetic radiation. In some example embodiments, the method can further include removing the dressing from the tissue site when the switchable adhesive has the third tack.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2 is a perspective view of an example embodiment of a dressing and a dressing interface of the therapy system of FIG. 1;

FIG. 3 is an exploded view of the dressing of FIG. 2;

FIG. 4 is a cross-sectional view of the dressing of FIG. 2 taken along line 4-4;

FIG. 5 is a perspective view of the dressing interface of FIG. 2;

FIG. 6 is a cross-sectional view of the dressing interface of FIG. 5 taken along line 6-6;

FIG. 7 is a bottom view of the dressing interface of FIG. 5;

FIG. 8 is a cross-sectional view with a portion shown in elevation of the therapy system of FIG. 1 in operation at a tissue site;

FIG. 9 is a block diagram illustrating operational steps for a process of treating a tissue site with the therapy system of FIG. 1; and

FIG. 10 is a block diagram illustrating operational steps of another example process for treating a tissue site with the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

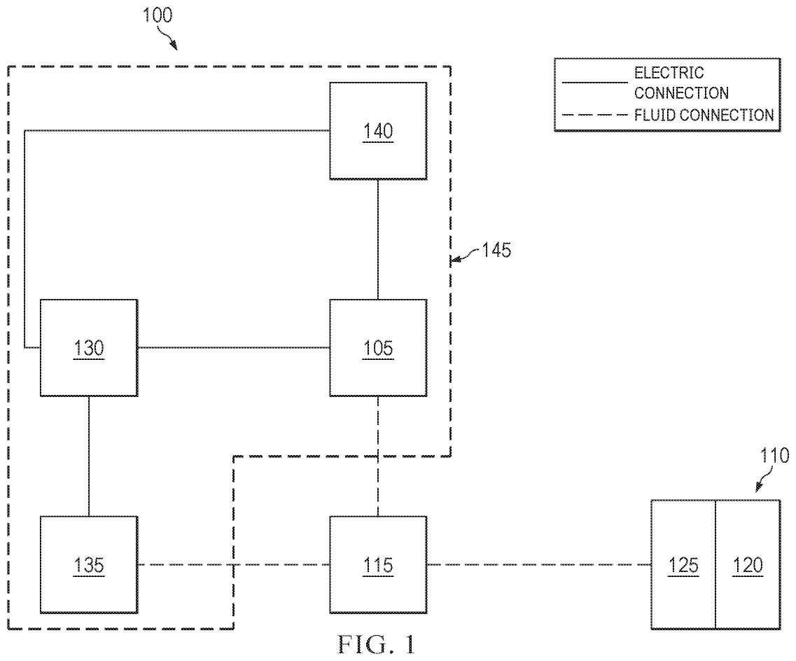

FIG. 1 is a block diagram of an example embodiment of a therapy system 100 that can provide negative-pressure therapy to a tissue site in accordance with this specification.

The term “tissue site” in this context broadly refers to a wound, surgical site incision, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and INSPIRE®2301 and INSPIRE®2327 polyurethane films, commercially available from Exopack Advanced Coatings, Wrexham, United Kingdom. In some embodiments, the cover 125 may comprise INSPIRE® 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

To ensure that a cover can maintain a sealed therapeutic environment over a tissue site, the attachment device may comprise an adhesive having high tackiness, high bond strength, or both high tackiness and high bond strength. A high bond strength adhesive is an adhesive that may readily bond to tissue and provide a seal between the cover and the tissue site. Tack describes a property of an adhesive that relates a particular bond strength of an adhesive on a substrate to a time period necessary to achieve the particular bond strength.

Occasionally, low level dressing leaks may occur between the cover and the epidermis surrounding a tissue site if the cover fails to completely seal to the epidermis. Covers are a balance between the strength of the adhesive required to enable the cover to seal against leaks and the pain which may result when the cover is removed. A high bond strength adhesive may be better for sealing, but the adhesive strength may cause significantly more discomfort upon cover removal. In addition, removing a cover with a bonding adhesive may cause significant damage to patients having delicate or damaged skin.

The dressing 110 may address these and other issue by providing a cover having a high bond strength adhesive and at least one electromagnetic transmission layer or surface, such as a light guide. The high bond strength adhesive may be switchable, i.e., capable of being switched between a first bond strength and a second bond strength. In some embodiments, the high bond strength adhesive can be switched in response to exposure to electromagnetic radiation, such as light. The dressing 110 may further include a dressing connector configured to provide both negative pressure to the tissue site and electromagnetic radiation to the electromagnetic transmission layer. At dressing removal, electromagnetic radiation provided to the electromagnetic transmission layer may cause the high bond strength adhesive to switch from a first bond strength to a second bond strength that is less than the first bond strength. The second bond strength may make removal of the dressing 110 more comfortable for a patient and decrease any risk that the adhesive may damage tissue.

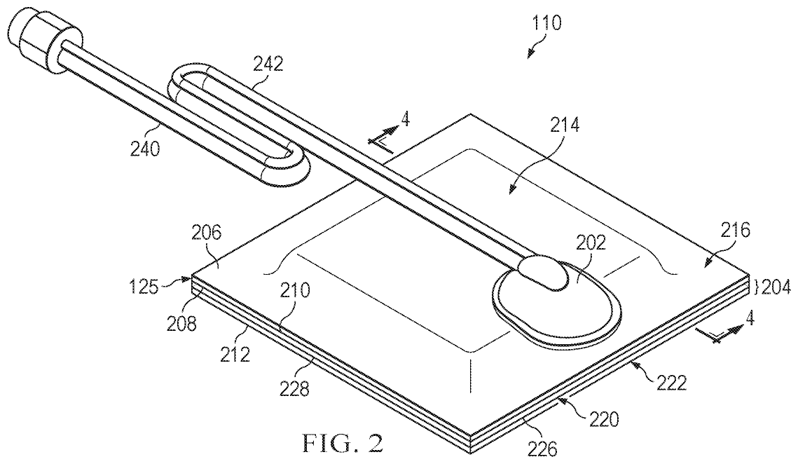

Referring to FIG. 2, a perspective view of an example embodiment of the dressing 110 and a dressing interface 202 that may be associated with the therapy system 100 is shown. In some embodiments, the dressing 110 may include a sealing member, such as the cover 125, a manifold, such as the tissue interface 120, and distribution layer, such as a wound contact layer 204. The cover 125 may have a first surface 206 and a second surface 208 opposite the first surface 206, a central portion 214, and a periphery 216. The periphery 216 may surround the central portion 214. In some embodiments, the dressing interface 202 may be configured to couple to the first surface 206 of the cover 125.

The wound contact layer 204 may include a first surface 210 and a second surface 212 opposite the first surface 210. In some embodiments, the second surface 208 of the cover 125 may be positioned adjacent to the first surface 210 of the wound contact layer 204. The second surface 208 of the cover 125 can also be coupled to the first surface 210 of the wound contact layer 204. If the dressing 110 is deployed at a tissue site, the second surface 212 of the wound contact layer 204 may be positioned adjacent to the tissue site. In some embodiments, the wound contact layer 204 may be configured to couple to the tissue site or tissue surrounding the tissue site.

In some embodiments, the cover 125 may be configured to block electromagnetic radiation from at least a portion of the electromagnetic radiation spectrum from penetrating through the cover 125. For example, in embodiments where the cover 125 includes a film such as polypropylene, polyurethane, or a similar material, pigment may be incorporated into the cover 125 such that electromagnetic radiation from at least a portion of the electromagnetic spectrum is blocked from penetrating through the cover 125. Additionally or alternatively, the cover 125 may include a nonwoven material or an aluminized foil film that may block electromagnetic radiation from at least a portion of the electromagnetic spectrum from penetrating through the cover 125. In some embodiments, the cover 125 may be separable from the remaining components of the dressing 110. For example, the cover 125 may be coupled to the remaining components of the dressing 110 with a releasable adhesive such that the cover 125 may be removed from the remaining components of the dressing 110 without leaving any adhesive residue on the remaining components of the dressing 110. If the cover 125 is removed from the dressing 110, electromagnetic radiation may penetrate into or through the remaining components of the dressing 110.

In some embodiments, the wound contact layer 204 may include more than one layer. For example, the wound contact layer 204 may include an electromagnetic transmission layer, such as a lightguide 220 and an adhesive layer, such as a switchable adhesive 222. The lightguide 220 may include the first surface 210 of the wound contact layer 204. The lightguide 220 may have a second surface 226 opposite the first surface 210. The switchable adhesive 222 may have a first surface 228 that may be coupled to the second surface 226 of the lightguide 220. The switchable adhesive 222 may include the second surface 212 of the wound contact layer 204. The second surface 212 may be opposite the first surface 228. In some embodiments, the lightguide 220 and the switchable adhesive 222 may be opposing surfaces of the wound contact layer 204 such that the lightguide 220 is the first surface 210 of the wound contact layer 204 and the switchable adhesive 222 is the second surface 212 of the wound contact layer 204.

In some embodiments, a release liner may be disposed adjacent to the second surface 212 of the wound contact layer 204. The release liner may be coupled to the second surface 212 of the wound contact layer 204. The release liner can provide rigidity to the dressing 110 prior to application of the dressing 110 at the tissue site. The release liner can also aid in the application of the dressing 110 onto the tissue site. The release liner can be peeled off or otherwise removed to expose the second surface 212 of the wound contact layer 204 before applying the dressing 110 to the tissue site.

The therapy system 100 may also include one or more fluid conductors, such as conduits that may be configured to couple the dressing interface 202 to the therapy unit 145. In some embodiments, the one or more conduits may include a negative-pressure conduit 240 and a fiber-optic cable 242. The negative-pressure conduit 240 may be configured to fluidly couple the therapy unit 145 to the dressing interface 202 to draw fluid from the dressing 110 with the negative-pressure source 105 and generate negative pressure at the dressing 110. The fiber-optic cable 242 may be configured to provide a pathway for a signal or electromagnetic radiation from the therapy unit 145 to the dressing interface 202. In some embodiments, electromagnetic radiation may be generated by one or more LEDs or another electromagnetic radiation source of the therapy unit 145. In some embodiments, a flat lightguide form factor may be configured to couple an electromagnetic radiation source of the therapy unit 145 to the dressing interface 202 in addition to or in place of the fiber-optic cable 242.

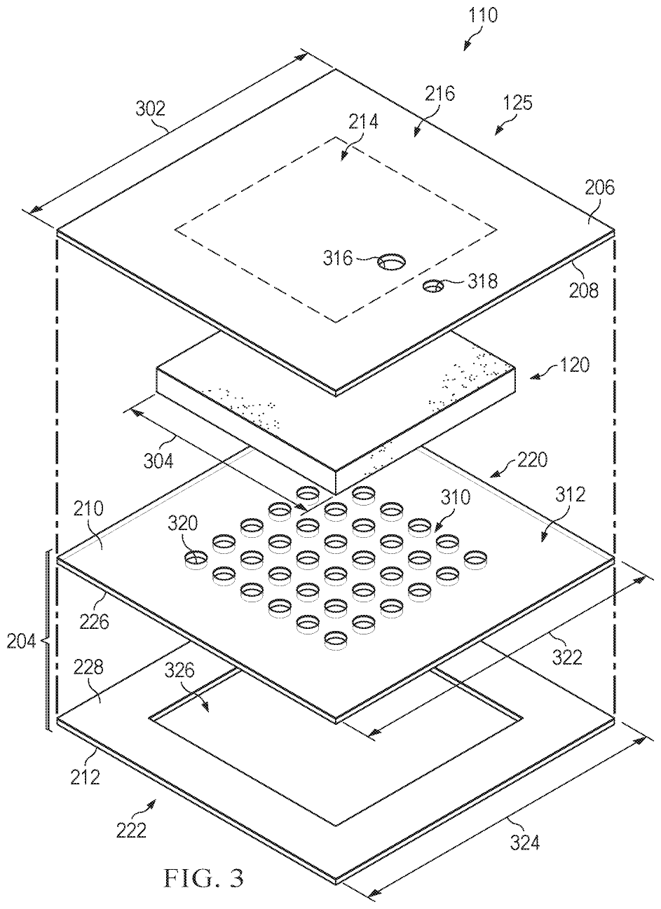

FIG. 3 is an exploded view of the dressing 110 of FIG. 2 illustrating additional details that may be associated with some embodiments. As shown in FIG. 3, the cover 125 may be substantially square and may have equal sides having a length 302. In other embodiments, the cover 125 may have other shapes, for example, polygonal, circular, ovular, amorphous shaped. The cover 125 may include a first aperture 316 and a second aperture 318. The first aperture 316 may be disposed through the central portion 214 of the cover 125. The second aperture 318 may be disposed through the periphery 216 of the cover 125.

The switchable adhesive 222 may be substantially square and may have equal sides having a length 324. In other embodiments, the switchable adhesive 222 may have other shapes, for example, polygonal, circular, ovular, amorphous shaped.

In some embodiments, the tissue interface 120 may be substantially square and may have equal sides having a length 304. In other embodiments, the tissue interface 120 may have other shapes, for example, polygonal, circular, ovular, amorphous shaped. The tissue interface 120 may be disposed between the second surface 208 of the cover 125 and the first surface 210 of the lightguide 220. In some embodiments, a surface of the tissue interface 120 facing the cover 125 may have a smaller area than the second surface 208 of the cover 125. Similarly, a surface of the tissue interface 120 facing the wound contact layer 204 may have an arca that is less than an area of the first surface 210 of the wound contact layer 204. In some embodiments, the tissue interface 120 may be disposed proximate to the central portion 214 of the cover 125.

The lightguide 220 may be substantially square and have equal sides having a length 322. In other embodiments, the lightguide 220 may have other shapes, for example, polygonal, circular, ovular, amorphous shaped. The lightguide 220 may have a central portion 310 and a peripheral portion 312. In some embodiments, the central portion 310 may be surround by the peripheral portion 312. The tissue interface 120 may be disposed between the central portion 214 of the cover 125 and the central portion 310 of the lightguide 220. The periphery 216 of the cover 125 may couple to the peripheral portion 312 of the lightguide 220 such that the periphery 216 of the cover 125 and the peripheral portion 312 of the lightguide 220 enclose the tissue interface 120.

In some embodiments, the lightguide 220 may include a plurality of apertures 320. The plurality of apertures 320 may be disposed in the central portion 310 of the lightguide 220 and may extend through the lightguide 220 from the first surface 210 to the second surface 226. In some embodiments, the plurality of apertures 320 may have a same diameter. In other embodiments, the plurality of apertures 320 may have different diameters. For example, the plurality of apertures 320 may have apertures 320 having a larger diameter preferentially positioned to encourage fluid flow in particular areas of the lightguide 220. In some embodiments, the plurality of apertures 320 are equidistantly spaced from each other and arranged in an array in the central portion 310. In other embodiments, the apertures 320 may be preferentially positioned in particular areas of the central portion 310. The plurality of apertures 320 may enable negative pressure from the negative-pressure source 105 to be distributed through the tissue interface 120, through the lightguide 220, and to a tissue site. In some embodiments, the plurality of apertures 320 may further provide a fluid path between the tissue site and the tissue interface 120.

The lightguide 220 may be configured to distribute electromagnetic radiation. In some embodiments, the lightguide 220 may be exposed on an edge or surface to electromagnetic radiation. The electromagnetic radiation may be transmitted through the lightguide 220 so that the electromagnetic radiation is transmitted from another edge or surface of the lightguide 220. In some embodiments, the electromagnetic radiation may be distributed so that the electromagnetic radiation is distributed to all surfaces of the lightguide 220. In some embodiments, the lightguide 220 may include a flexible and compressible polymer such as a silicone. In other embodiments, the lightguide 220 may be comprised of a mesh of flexible, plastic/polymer optical fiber mono-filaments that are bonded to the switchable adhesive 222. The lightguide 220 may be treated such that electromagnetic radiation can be diffused over the entire area of the lightguide 220. In some embodiments, electromagnetic radiation may contact the lightguide 220 and may be refracted over the entirety of the lightguide 220 and may be reflected through the lightguide 220. Alternatively, electromagnetic radiation may be internally reflected through the lightguide 220. The lightguide 220 may be porous or breathable to allow for electromagnetic radiation and/or fluid communication between the switchable adhesive 222 and the lightguide 220.

In some embodiments, the switchable adhesive 222 may comprise a layer of adhesive that is configured to transition from a first tack to a second tack. The switchable adhesive 222 may have a first tack before the switchable adhesive 222 is exposed to electromagnetic radiation. And the switchable adhesive 222 may have a second tack after the switchable adhesive 222 is exposed to electromagnetic radiation. For example, the switchable adhesive 222 may transition from the first tack to the second tack in response to electromagnetic radiation having a first wavelength, i.e., a first electromagnetic radiation. In some embodiments, the second tack may be less than the first tack. For example, the first tack may be selected to secure the dressing 110 to a tissue site, and the second tack may be selected to weaken the switchable adhesive to permit removal of the dressing 110.

In other embodiments, the switchable adhesive 222 may be a dual switchable adhesive such that the switchable adhesive 222 is configured to have three or more phases. The switchable adhesive 222 may have a first tack before the switchable adhesive is exposed to an electromagnetic radiation. The switchable adhesive 222 may be configured to switch to a second tack upon exposure to the first electromagnetic radiation. The first electromagnetic radiation may cure the switchable adhesive 222 from the first tack to the second tack. The second tack may be greater than the first tack. In some embodiments, the switchable adhesive 222 may further be configured to switch from the second tack to a third tack upon exposure to an electromagnetic radiation of a second wavelength, i.e., a second electromagnetic radiation. The third tack may be less than the second tack. The first electromagnetic radiation and the second electromagnetic radiation necessary to switch the switchable adhesive 222 may be determined based on the material of the switchable adhesive 222. For example, in some embodiments the first electromagnetic radiation and/or the second electromagnetic radiation may be infrared wavelengths between about 650 nm and about 850 nm that may cause the switchable adhesive 222 to switch from the first tack to the second tack or from the second tack to the third tack. In other embodiments, the first electromagnetic radiation and/or the second electromagnetic radiation may be ultraviolet wavelengths between about 200 nm and about 450 nm that may cause the switchable adhesive 222 to switch from the first tack to the second tack or from the second tack to the third tack. In some embodiments, the first electromagnetic radiation and the second electromagnetic radiation may be different wavelengths that may be configured to cause the switchable adhesive 222 to switch from the first tack to the second tack or from the second tack to the third tack.

In embodiments where the switchable adhesive is a dual switchable adhesive, the first tack may enable a health care provider or a user to reposition the dressing 110 at a tissue site until the dressing 110 is in an optimal position at the tissue site. The second tack may be greater than the first tack such that the dressing 110 is secured in place at the tissue site with the second tack. The third tack may be less than the second tack such that the dressing 110 may be easily removable from the tissue site when the switchable adhesive has the third tack.

In some embodiments, the switchable adhesive 222 may have a central aperture 326 such that the second surface 226 of the central portion 310 of the lightguide 220 does not contact the switchable adhesive 222. The central aperture 326 may enable fluid from a tissue site to travel from the tissue site through the plurality of apertures 320 of the lightguide to the tissue interface 120. In other embodiments, the switchable adhesive 222 may not include the central aperture 326 but may include a plurality of apertures (not pictured) in a central portion of the switchable adhesive 222 that may be configured to enable fluid from the tissue site to travel from the tissue site through the plurality of apertures of the lightguide to the tissue interface 120. In some embodiments, the central aperture 326 or the plurality of apertures may enable electromagnetic radiation to reach the tissue site. For example, the therapy system 100 may be configured to treat the tissue site with electromagnetic radiation having a third wavelength, i.e., a third electromagnetic radiation. The third electromagnetic radiation may be a therapeutic electromagnetic radiation that may be configured to treat the tissue site.

The switchable adhesive 222 may include one or more materials including, but not limited to, polyurethane, acrylic (e.g., cyanoacrylate), hydrogel, silicon or silicone-based material, natural rubber, synthetic rubber, styrene block copolymers, polyvinyl ethers, poly (meth) acrylates, polyolefins, hydrocolloid (e.g., a rubber-based hydrocolloid), or a combination thereof. In some embodiments, the switchable adhesive 222 may include a polymer or co-polymer. For example, the switchable adhesive 222 may include a copolymer of polyurethane and silicone or various acrylic co-polymers.

In some example embodiments, the switchable adhesive 222 may include an alkoxy acrylic dual cure system. The switchable adhesive 222 may include an unsaturated acrylate group that may be configured to react with electromagnetic radiation in the UV spectrum to undergo chain-growth polymerization. More specifically, the UV electromagnetic radiation may link urethane with acrylate such that the unsaturated acrylate functional group polymerizes responsive to reactions caused by photo initiators. The photo initiators can generate free radicals and initiate chain-growth polymerization which may switch the switchable adhesive 222 between a first tack and a second tack or a second tack and a third tack.

In embodiments of the switchable adhesive 222 being a single switchable adhesive, the first tack may have a peel strength of greater than about 8N/25 mm on stainless steel at an angle of 180 degrees at room temperature. For example, the first tack of the switchable adhesive 222 may have a peel strength of greater than about 15N/25 mm on stainless steel at an angle of 180 degrees at room temperature, or the first tack of the switchable adhesive 222 may have a peel strength between about 10N/25 mm and about 15N/25 mm on stainless steel at an angle of 180 degrees at room temperature. The second tack of the switchable adhesive 222 may have a peel strength of less than about 7N/25 mm on stainless steel at an angle of 180 degrees at room temperature. For example, the second tack of the switchable adhesive 222 may have a peel strength between about 3N/25 mm and about 6N/25 mm on stainless steel at an angle of 180 degrees at room temperature.

In embodiments of the switchable adhesive 222 being a dual switchable adhesive, the first tack may have a peel strength of less than about 7N/25 mm on stainless steel at an angle of 180 degrees at room temperature. For example, the first tack of the switchable adhesive 222 may have a peel strength between about 4N/25 mm and about 6N/25 mm on stainless steel at an angle of 180 degrees at room temperature. The second tack of the switchable adhesive 222 may have a peel strength of greater than about 8N/25 mm on stainless steel at an angle of 180 degrees at room temperature. For example, the second tack of the switchable adhesive 222 may have a peel strength of greater than about 15N/25 mm on stainless steel at an angle of 180 degrees at room temperature, or the second tack of the switchable adhesive 222 may have a peel strength between about 10N/25 mm and about 15N/25 mm on stainless steel at an angle of 180 degrees at room temperature. Additionally, or alternatively, the third tack of the switchable adhesive 222 may have a peel strength of less than about 7N/25 mm on stainless steel at an angle of 180 degrees at room temperature. For example, the third tack of the switchable adhesive 222 may have a peel strength between about 3N/25 mm and about 6N/25 mm on stainless steel at an angle of 180 degrees at room temperature.

In some embodiments, the dressing 110 may be substantially square in shape. In other embodiments, the dressing 110 may be rectangular, circular, another shape, or cut to fit a specific sized tissue site. In some embodiments, each of the cover 125, the tissue interface 120, the lightguide 220, and the switchable adhesive 222 may a same shape. In other embodiments, the cover 125, the tissue interface 120, the lightguide 220, and the switchable adhesive 222 may be shaped differently than each other. In some embodiments, the length 302, the length 322, and the length 324 may be the same. In other embodiments, the length 302, the length 322, and the length 324 may be different. Preferably, the length 304 may be less than the length 302, the length 322, and the length 324.

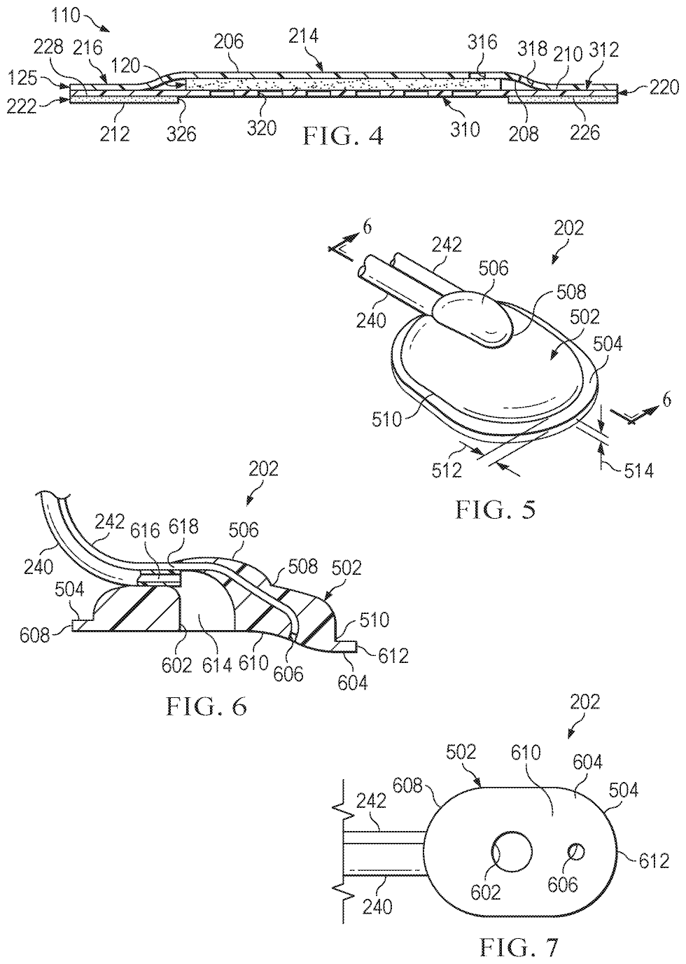

FIG. 4 is a sectional view of the dressing 110 illustrating additional details that may be associated with some embodiments. As illustrated in FIG. 4, the switchable adhesive 222 may be coupled to the lightguide 220. The tissue interface 120 may be positioned adjacent to the plurality of apertures 320 in the central portion 310 of the lightguide 220. The cover 125 can be positioned adjacent to the lightguide 220 and the periphery 216 of the cover 125 can be coupled to the peripheral portion 312 of the lightguide 220. The tissue interface 120 is disposed between the central portion 214 of the cover 125 and the central portion 310 of the lightguide 220. In some embodiments, the tissue interface 120 is encapsulated by the cover 125 and the lightguide 220. The first aperture 316 of the cover 125 is disposed in the central portion 214 of the cover 125. In some embodiments, the first aperture 316 provides fluid communication across the cover 125 from the first surface 206 of the cover 125 to the second surface 208 of the tissue interface 120. The first aperture 316 may be in fluid communication with the tissue interface 120. The second aperture 318 of the cover 125 is disposed in the periphery 216 of the cover 125. In some embodiments, the second aperture 318 extends across the cover 125 from the first surface 206 of the cover 125 to the second surface 208 of the cover 125. In some embodiments, the second aperture 318 provides access to the lightguide 220. For example, the second aperture 318 may permit transmission of electromagnetic radiation across the cover 125 with the lightguide 220.

FIG. 5 is a perspective view of the dressing interface 202 illustrating additional details that may be associated with some embodiments. The dressing interface 202 may have a connector body 502, a connector flange 504, and a connector port 506. The connector body 502 may be a spherical cap or other semi-spheroid body having an apex 508 and an edge 510. In some embodiments, the edge 510 may located at a diameter of the connector body 502. In other embodiments, the connector body 502 may have a semi-ellipsoid or semi-ovular shape. In still other embodiments, the connector body 502 may be polygonal or amorphous shaped.

The connector flange 504 may be coupled to the connector body 502 at the edge 510. The connector flange 504 may extend from the connector body 502 a distance 512 and have a thickness 514. The connector port 506 may be coupled to the connector body 502 proximate to the apex 508 of the connector body 502. In some embodiments, the connector port 506 may be configured to receive the negative-pressure conduit 240. The connector port 506 may be configured to seal to the negative-pressure conduit 240 and fluidly couple the negative-pressure conduit 240 to the dressing interface 202. The connector port 506 may also be configured to receive the fiber-optic cable 242. The connector port 506 may be configured to seal to the fiber-optic cable 242 and to couple the fiber-optic cable 242 to the dressing interface 202. In some embodiments, the negative-pressure conduit 240 and the fiber-optic cable 242 may be integral components of the dressing interface 202. In other embodiments, the negative-pressure conduit 240 and the fiber-optic cable 242 may be separable from the dressing interface 202.

FIG. 6 is a sectional view of the dressing interface 202 taken along line 6-6 and illustrating additional details that may be associated with some embodiments. As illustrated in FIG. 6, the dressing interface 202 may include a first opening 602, a coupling surface 604, and a second opening 606. In some embodiments, the connector body 502 may a substantially solid body. In other embodiments, the connector body 502 may comprise a dome having a concave surface forming a cavity opposite the connector port 506.

In some embodiments, the coupling surface 604 may be opposite the apex 508 of the connector body 502. The coupling surface 604 may be substantially flat. In other embodiments, the coupling surface 604 may include one or more contours configured to engage a mating surface of another body. The dressing interface 202 may have a first end 608 and a second end 612. The first end 608 may be proximate to a the negative-pressure conduit 240, and the second end 612 may be opposite the first end 608. In some embodiments, the coupling surface 604 may extend from the first end 608 toward the second end 612. The coupling surface 604 may be disposed in a first plane from the first end 608 to a location 610 between the first end 608 and the second end 612. A portion of the coupling surface 604 may be disposed in a second plane extending from the location 610 toward the second end 612. In some embodiments, the second plane may intersect the first plane, causing the coupling surface 604 to slope from the location 610 to the second end 612. In other embodiments, the coupling surface 604 may curve the location 610 to the second end 612. The coupling surface 604 may have varying topography so that the coupling surface 604 may accommodate changes in topography to the cover 125 and is able to couple to the dressing 110 such that the first opening 602 may couple with the first aperture 316 and the second opening 606 may couple with the second aperture 318.

The connector port 506 may include negative-pressure opening 616 and a cable opening 618. The negative-pressure opening 616 may receive the negative-pressure conduit 240 and fluidly couple a lumen of the negative-pressure conduit 240 to the dressing interface 202. The first opening 602 may be disposed between the first end 608 and the location 610. In some embodiments, the first opening 602 may be disposed in the coupling surface 604 proximate to the connector port 506. The first opening 602 may depend into the connector body 502 to form a fluid passage 614. The fluid passage 614 may fluidly couple the first opening 602 to the negative-pressure opening 616 of the connector port 506, permitting fluid communication through the dressing interface 202.

The cable opening 618 may receive the fiber-optic cable 242 to communicatively couple the fiber-optic cable 242 to the dressing interface 202. The second opening 606 may be disposed between the location 610 and the second end 612. In some embodiments, the second opening 606 may depend into the connector body 502 to form a channel 620. The channel 620 may communicatively couple the second opening 606 to the cable opening 618 of the connector port 506, permitting communication through the dressing interface 202.

In some embodiments, the first opening 602 may be configured to be disposed adjacent to the first aperture 316 of the cover 125 and the second opening 606 may be configured to be disposed adjacent to the second aperture 318 of the cover 125. The first opening 602 may be configured to fluidly couple the dressing interface 202 to the tissue interface 120 through the first aperture 316, and the second opening 606 may be configured to communicatively couple the dressing interface 202 to the lightguide 220 through the second aperture 318.

FIG. 7 is a bottom view of the dressing interface 202 illustrating additional details that may be associated with some embodiments. The first opening 602 and the second opening 606 are visible on the coupling surface 604 of the dressing interface 202. In some embodiments, the first opening 602 may be disposed proximate to a center of the coupling surface 604. The second opening 606 may be disposed between the first opening 602 and the second end 612. In some embodiments, the first opening 602 may have an effective diameter that is greater than an effective diameter of the second opening 606.

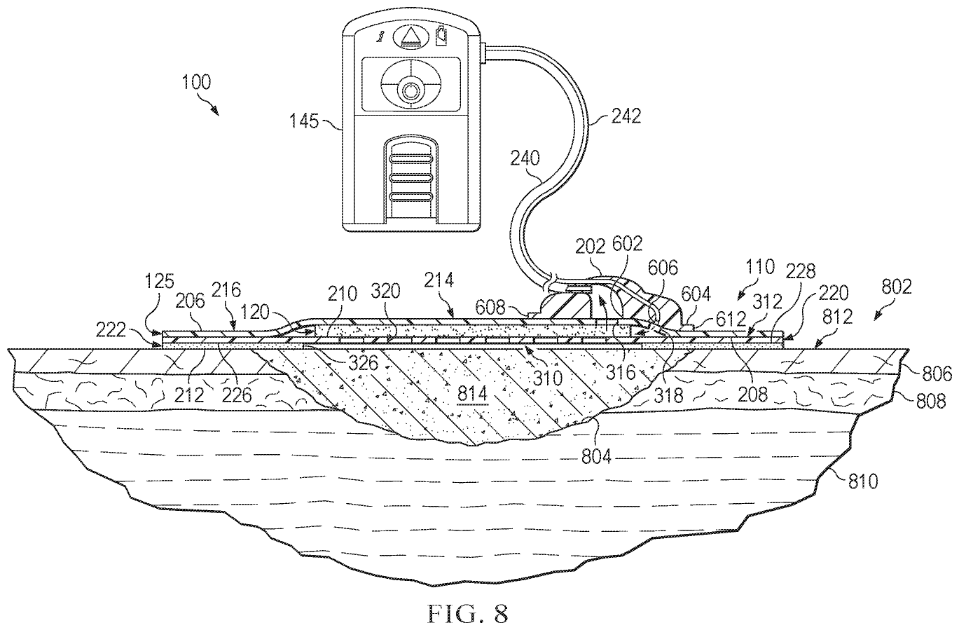

FIG. 8 is a sectional view with a portion shown in elevation of the therapy system 100 illustrating additional details that may be associated with some embodiments. In some embodiments, the dressing 110 may be disposed over a tissue site 802 to cover a wound 804. The tissue site 802 may be or may include a defect or targeted treatment site, such as the wound 804, which may be partially or completely filled or covered by the dressing 110. The wound 804 may be in an epidermis 806. In some examples, the wound 804 may extend through the epidermis 806 and into a dermis 808. In other examples, the wound 804 may extend through the epidermis 806 and the dermis 808 into a subcutaneous tissue 810. In some embodiments, the second surface 212 of the wound contact layer 204 may be brought into contact with a portion of the epidermis 806 surrounding the wound 804, such as a periwound region 812 as well as the wound 804. In some embodiments, a tissue interface, such as a manifold 814 can be disposed in the wound 804 prior to placement of the dressing 110 at the tissue site 802.

In some embodiments, the dressing 110 can be positioned over the wound 804 and the manifold 814. For example, the switchable adhesive 222 may be adjacent to the periwound region 812 and the central portion 310 of the lightguide 220 may be adjacent to a surface of the manifold 814. The central portion 310 of the lightguide 220 having the plurality of apertures 320 may be proximate to and in fluid communication with the manifold 814. The tissue interface 120 may be fluidly coupled to the manifold 814 and the wound 804 through the plurality of apertures 320 of the lightguide 220 and the switchable adhesive 222. The therapy unit 145 may be fluidly coupled to the tissue interface 120 and the manifold 814 through the dressing interface 202 and the negative-pressure conduit 240. For example, the dressing interface 202 may couple to the cover 125 such that the first aperture 316 and the second aperture 318 are fluidly coupled to the therapy unit 145 through the dressing interface 202. The therapy unit 145 may be communicatively coupled to the lightguide 220 through the dressing interface 202 and the fiber-optic cable 242.

In operation, fluid may be drawn from the manifold 814 by the therapy unit 145, generating a negative pressure proximate to the wound 804. Fluid may be removed from the manifold 814 by the negative-pressure source 105 of the therapy unit 145 through the dressing 110, the dressing interface 202 and the negative-pressure conduit 240. The fluid may travel from the manifold 814, through the central aperture 326 of the switchable adhesive 222, through the plurality of apertures 320 of the lightguide 220, the tissue interface 120, and the first aperture 316 of the cover 125. The fluid may then flow through the first opening 602 of the dressing interface 202 and travel through the negative-pressure conduit 240 to reach the therapy unit 145 which may include the container 115 in some embodiments. In other embodiments, the fluid may not be removed from the dressing 110 but may be absorbed by the dressing 110 after the fluid is drawn from the wound 804, through the manifold 814, through the central aperture 326 of the switchable adhesive 222, and through the plurality of apertures 320 of the lightguide 220.

Electromagnetic radiation may be provided to the dressing 110 by an electromagnetic radiation source such as LEDs disposed in the therapy unit 145. In some embodiments, the electromagnetic radiation source may be another source that may be configured to generate electromagnetic radiation of different wavelengths that may be transmitted by the fiber-optic cable 242 through the dressing interface 202 to the lightguide 220. The electromagnetic radiation may be communicated from the therapy unit 145 through the fiber-optic cable 242 to the dressing interface 202. The electromagnetic radiation may be communicated through the dressing interface 202 and from the second opening 606 through the second aperture 318 of the cover 125 to reach the lightguide 220. In other embodiments, the electromagnetic radiation source may be disposed within the dressing interface 202 or within the dressing 110 proximate to the lightguide 220. The electromagnetic radiation source may be communicatively coupled to the therapy unit 145 which may enable the electromagnetic radiation source to be controlled to deliver a desired electromagnetic radiation to the dressing 110. The electromagnetic radiation may be refracted through the lightguide 220 dispersing the electromagnetic radiation to the first surface 228 of the switchable adhesive 222. In some embodiments, a substantial portion of the first surface 228 of the switchable adhesive 222 may be exposed to the electromagnetic radiation. The electromagnetic radiation may have a wavelength configured to cure the switchable adhesive 222, switch the switchable adhesive 222 from one tack to another tack. In some embodiments, the wavelength of the electromagnetic radiation may provide a therapeutic electromagnetic radiation to the wound 804.

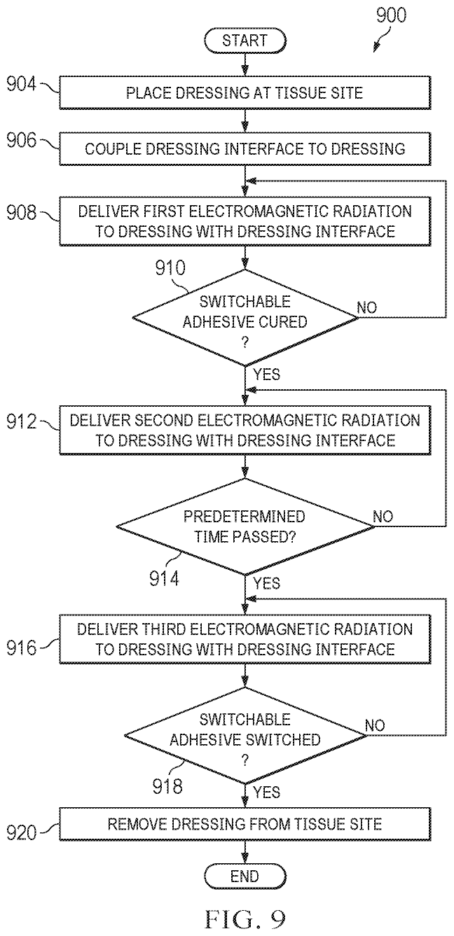

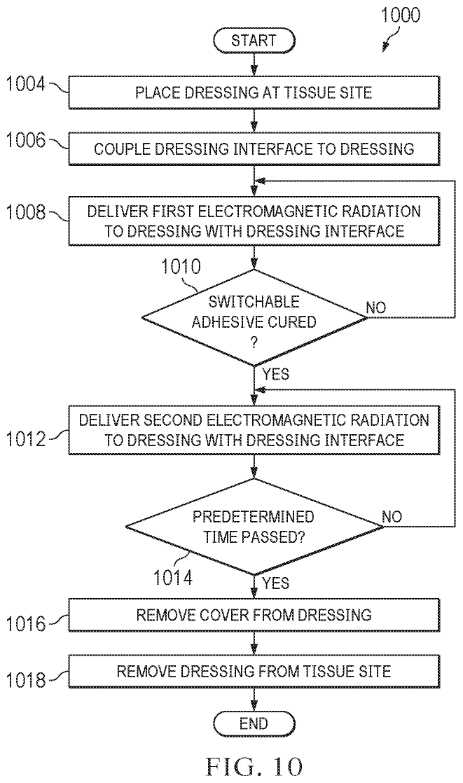

FIG. 9 is a flow chart 900 illustrating operational steps of a process that may be implemented by the therapy system 100. The process may begin at block 904 where a dressing can be positioned at tissue site. For example, the dressing 110 can be placed at the tissue site 802. In some embodiments, placement of the dressing 110 may be proceeded by placement of the manifold 814. The switchable adhesive 222 may have the first tack, permitting the dressing 110 to be moved and repositioned if desired. The process continues at block 906, where a dressing interface is coupled to the dressing. For example, the dressing interface 202 may be coupled to the dressing 110. Preferably, the first opening 602 of the dressing interface 202 may be aligned with the first aperture 316 of the cover 125, and the second opening 606 of the dressing interface 202 may be aligned with the second aperture 318 of the cover 125. In this configuration, the first opening 602 may be in fluid communication with to the tissue interface 120 and the second opening 606 of the dressing interface 202 may be communicatively coupled to the peripheral portion 312 of the lightguide 220 of the wound contact layer 204.

The process continues at block 908 where a first electromagnetic radiation is delivered to the dressing. For example, the therapy unit 145 may generate the first electromagnetic radiation using LEDs. The first electromagnetic radiation may be transmitted from the therapy unit 145 through the fiber-optic cable 242 to the dressing interface 202. The first electromagnetic radiation may be transmitted from the dressing interface 202 through the second aperture 318 of the cover 125 to the lightguide 220 of the wound contact layer 204. The first electromagnetic radiation may be refracted through the lightguide 220 such that the first electromagnetic radiation is reflected towards and dispersed to a substantial portion of the first surface 228 of the switchable adhesive 222. The process continues at decisional block 910 where the process determines if the switchable adhesive is cured. For example, the dressing 110 may be tested to determine if the switchable adhesive 222 is cured. In some embodiments, a health care provider or a user may attempt to lift a corner or another part of the dressing 110 to determine if the dressing 110 can be lifted. If the dressing lifts the process continues on the NO path to block 908 and the first electromagnetic radiation is applied.

If the dressing does not lift, the process continues on the YES path to block 912 where a second electromagnetic radiation is delivered to the wound contact layer of the dressing through the dressing interface. For example, the controller 130 of the therapy unit 145 may generate the second electromagnetic radiation with, for example LEDs. The second electromagnetic radiation may have a wavelength that is different than the wavelength of the first electromagnetic radiation. In some embodiments, the second electromagnetic radiation may be a therapeutic electromagnetic radiation that may be used to provide a therapy.

In some embodiments, following transition of the switchable adhesive 222 from the first tack to the second tack, the controller 130 may operate the negative-pressure source 105 to generate negative pressure at the dressing 110. Negative pressure may be supplied to the dressing 110 if the switchable adhesive 222 has the second tack. For example, the negative-pressure source 105 may supply negative pressure to the dressing 110 before the second electromagnetic radiation is delivered to the wound contact layer 204 but after the switchable adhesive 222 has the second tack, during the application of the second electromagnetic radiation to the wound contact layer 204, or after the application of the second electromagnetic radiation to the wound contact layer 204 but before application of the third electromagnetic radiation to the wound contact layer 204.

The process continues to decisional block 914 where the process determines whether a predetermined amount of time has passed. For example, the controller 130 may start a clock following application of the second electromagnetic radiation. The controller 130 can determine if the predetermined time is reached by the clock. In some embodiments, the predetermined time may correspond to the time that the dressing 110 should be applied to a tissue site. In other embodiments, the predetermined time may correspond to the time that the second electromagnetic radiation should be applied. If the predetermined time is not reached, the process follows the NO path to block 912 to deliver the second electromagnetic radiation.

If the predetermined time is reached, the process follows the YES path to block 916, where a third electromagnetic radiation may be transmitted. For example, the controller 130 of the therapy unit 145 may operate LEDs to generate and transmit the third electromagnetic radiation to the wound contact layer 204 of the dressing 110 through the dressing interface 202. The third electromagnetic radiation may have a wavelength that is different from the first electromagnetic radiation and the second electromagnetic radiation. The third electromagnetic radiation may be configured to switch the switchable adhesive 222 from the second tack to a third tack. The third tack may be less than the second tack, permitting the dressing 110 to be removed with minimal force.

The process continues to decisional block 918. At decisional block 918 the process may determine if the switchable adhesive has switched from the second tack to the third tack. In some embodiments, a health care provider or a user may attempt to lift a corner or another part of the dressing 110 to determine if the dressing 110 can be moved. If the dressing 110 lifts, then the switchable adhesive 222 may be switched to the third tack. If the dressing 110 does not lift, then the switchable adhesive 222 may still have the second tack. If the switchable adhesive still has the second tack, the process follows the NO path to block 916 where the process continues to deliver the third electromagnetic radiation.

If the dressing lifts, the process follow the YES path to block 920 where the dressing is removed and the process ends. For example, the dressing 110 may be removed from the tissue site 802.

FIG. 10 is a flow chart 1000 illustrating operational steps of a process that may implemented by the therapy system 100. The process begins at block 1004 where a dressing is placed at a tissue site. For example, the dressing 110 can be placed at the tissue site 802. The switchable adhesive 222 may have the first tack, permitting the dressing 110 to be moved and repositioned if desired.

The process continues at block 1006 where a dressing interface can be coupled to the dressing. For example, the dressing interface 202 may be coupled to the dressing 110. Preferably, the first opening 602 of the dressing interface 202 may be aligned with the first aperture 316 of the cover 125, and the second opening 606 of the dressing interface 202 may be aligned with the second aperture 318 of the cover 125. In this configuration, the first opening 602 may be in fluid communication with to the tissue interface 120 and the second opening 606 of the dressing interface 202 may be communicatively coupled to the peripheral portion 312 of the lightguide 220 of the wound contact layer 204.

The process continues at block 1008 where a first electromagnetic radiation is delivered to the dressing. For example, the therapy unit 145 may generate the first electromagnetic radiation using LEDs. The first electromagnetic radiation may be transmitted from the therapy unit 145 through the fiber-optic cable 242 to the dressing interface 202. The first electromagnetic radiation may be transmitted from the dressing interface 202 through the second aperture 318 of the cover 125 to the lightguide 220 of the wound contact layer 204. The first electromagnetic radiation may be refracted through the lightguide 220 such that the first electromagnetic radiation is reflected towards and dispersed to a substantial portion of the first surface 228 of the switchable adhesive 222. The process continues at decisional block 1010 where the process determines if the switchable adhesive is cured. For example, the dressing 110 may be tested to determine if the switchable adhesive 222 is cured. In some embodiments, a health care provider or a user may attempt to lift a corner or another part of the dressing 110 to determine if the dressing 110 can be lifted. If the dressing lifts the process continues on the NO path to block 1008 and the first electromagnetic radiation is applied.

If the dressing does not lift, the process continues on the YES path to block 1012 where a second electromagnetic radiation is delivered to the wound contact layer of the dressing through the dressing interface. For example, the controller 130 of the therapy unit 145 may generate the second electromagnetic radiation with, for example LEDs. The second electromagnetic radiation may have a wavelength that is different than the wavelength of the first electromagnetic radiation. In some embodiments, the second electromagnetic radiation may be a therapeutic electromagnetic radiation that may be used to provide a therapy.

In some embodiments, following transition of the switchable adhesive 222 from the first tack to the second tack, the controller 130 may operate the negative-pressure source 105 generate negative pressure at the dressing 110. Negative pressure may be supplied to the dressing 110 if the switchable adhesive 222 has the second tack. For example, the negative-pressure source 105 may supply negative pressure to the dressing 110 before the second electromagnetic radiation is delivered to the wound contact layer 204 but after the switchable adhesive 222 has the second tack, during the application of the second electromagnetic radiation to the wound contact layer 204, or after the application of the second electromagnetic radiation to the wound contact layer 204 but before application of the third electromagnetic radiation to the wound contact layer 204.

The process continues to decisional block 1014 where the process determines whether a predetermined amount of time has passed. For example, the controller 130 may start a clock following application of the second electromagnetic radiation. The controller 130 can determine if the predetermined time is reached by the clock. In some embodiments, the predetermined time may correspond to the time that the dressing 110 should be applied to a tissue site. In other embodiments, the predetermined time may correspond to the time that the second electromagnetic radiation should be applied. If the predetermined is not reached, the process follows the NO path to block 1012 to deliver the second electromagnetic radiation.

If the predetermined time is reached, continues on the YES bath to block 1016, where the cover may be removed from the dressing. For example, the cover 125 may be removed from the dressing 110. As described above with reference to FIG. 2, the cover 125 may be configured to block ambient electromagnetic radiation from passing through the dressing 110. If the cover 125 is removed from the dressing 110, ambient electromagnetic radiation may reach the lightguide 220 of the dressing 110 and may be refracted through the lightguide 220. The ambient electromagnetic radiation may be reflected towards and dispersed by the lightguide 220 to a substantial portion of the first surface 228 of the switchable adhesive 222. The ambient electromagnetic radiation may be configured to switch the switchable adhesive 222 from the second tack to a third tack. The third tack may be less than the second tack such that the dressing 110 can be removed from the tissue site 802. The process continues to block 1018, where the dressing can be removed. For example, the dressing 110 may be removed from the tissue site 802.

The systems, apparatuses, and methods described herein may provide significant advantages. For example, the dressing 110 may allow a clinician or a health care provider the ability to reposition the dressing 110 as needed prior to the switchable adhesive 222 being cured to adhere the dressing 110 to the tissue site 802. The dressing interface 202 and the lightguide 220 may enable the switchable adhesive 222 to be cured from a first tack to a second tack and de-activated from the second tack to a third tack without the need of an external electromagnetic radiation source. The cover 125 may block ambient electromagnetic radiation from interfering with the switchable adhesive 222 such that any electromagnetic radiation that contacts the switchable adhesive 222 is directed by a health care provider or user from the therapy unit to the wound contact layer 204 of the dressing 110.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site with negative pressure, the system comprising:
a dressing comprising:
a cover configured to be disposed over the tissue site;
a tissue interface configured to be positioned adjacent to the cover; and
a wound contact layer configured to be positioned adjacent to the tissue interface; and
a dressing interface configured to be coupled to the cover to fluidly couple the tissue interface and the wound contact layer to a therapy unit and to transmit electromagnetic radiation through the cover.

2. The system of claim 1, wherein the cover is configured to block ambient electromagnetic radiation.

3. The system of claim 1, wherein the wound contact layer comprises:
a lightguide configured to receive and distribute the electromagnetic radiation from the dressing interface, the lightguide comprising a first surface of the wound contact layer; and
a switchable adhesive configured to transition between a first tack and a second tack in response to exposure to the electromagnetic radiation from the dressing interface, the switchable adhesive comprising a second surface of the wound contact layer, the second surface being opposite of the first surface.

4. The system of claim 3, wherein the lightguide is configured to distribute the electromagnetic radiation from the dressing interface to the switchable adhesive.

5. The system of claim 4, wherein the electromagnetic radiation comprises a first electromagnetic radiation and the switchable adhesive is further configured to transition from the second tack to a third tack in response to exposure to a second electromagnetic radiation from the dressing interface.

6. The system of claim 3, wherein the lightguide is further configured to distribute a therapeutic electromagnetic radiation to the tissue site.

7. The system of claim 1, wherein the wound contact layer further comprises a central portion and a periphery, the tissue interface configured to be positioned adjacent to the central portion of the wound contact layer.

8. The system of claim 7, wherein the dressing interface comprises a first opening and a second opening, the first opening configured to be fluidly coupled to the tissue interface and the second opening configured to couple to the periphery of the wound contact layer.

9. The system of claim 1, further comprising a fiber-optic cable configured to couple the dressing interface to the therapy unit.

10. The system of claim 1, wherein the cover is configured to be removable from the dressing.

11. A dressing for treating a tissue site, the dressing comprising:
a sealing member configured to be disposed over the tissue site;
a manifold configured to be positioned adjacent to the sealing member; and
a distribution layer comprising:
an electromagnetic transmission layer configured to receive and distribute electromagnetic radiation; and
an adhesive layer configured to transition between a first tack and a second tack in response to exposure to electromagnetic radiation distributed by the electromagnetic transmission layer.

12. A method of treating a tissue site, the method comprising:
placing a dressing adjacent to the tissue site, the dressing comprising:
a cover configured to be disposed over the tissue site;
a tissue interface configured to be positioned adjacent to the cover; and
a wound contact layer configured to be positioned adjacent to the tissue interface, the wound contact layer comprising:
a lightguide configured to receive and distribute electromagnetic radiation; and
a switchable adhesive configured to transition between a first tack and a second tack in response to exposure to the electromagnetic radiation;
coupling a dressing interface to the dressing, the dressing interface configured to deliver negative pressure and electromagnetic radiation to the dressing;
delivering a first electromagnetic radiation to the wound contact layer through the dressing interface; and
operating an electromagnetic radiation source to deliver a second electromagnetic radiation to the wound contact layer through the dressing interface.

13. The method of claim 12, wherein delivering the first electromagnetic radiation to the wound contact layer cures the switchable adhesive from the first tack to the second tack.

14. The method of claim 12, wherein the second electromagnetic radiation is a therapeutic electromagnetic radiation configured to treat the tissue site.

15. The method of claim 12, further comprising operating the electromagnetic radiation source to deliver a third electromagnetic radiation to the wound contact layer wherein the third electromagnetic radiation is configured to switch the switchable adhesive from the second tack to a third tack.

16. The method of claim 15, further comprising removing the dressing from the tissue site when the switchable adhesive has the third tack.

17. The method of claim 15, wherein the wound contact layer further comprises a central portion and a periphery, the tissue interface configured to be positioned adjacent to the central portion of the wound contact layer.

18. The method of claim 17, wherein coupling the dressing interface to the dressing comprises disposing a first opening of the dressing interface adjacent to the tissue interface and disposing a second opening of the dressing interface adjacent to the periphery of the wound contact layer.

19. The method of claim 12, further comprising removing the cover from the dressing to expose the dressing to ambient electromagnetic radiation, the switchable adhesive configured to switch from the second tack to a third tack when exposed to the ambient electromagnetic radiation.

20. The method of claim 19, further comprising removing the dressing from the tissue site when the switchable adhesive has the third tack.

21. (canceled)
